(19) United States
(12) Patent Application Publication
(10) Pub. No.: US 2021/0228148 A1
(43) Pub. Date: Jul. 29, 2021

(54) System and Method for Lesion Monitoring

(71) Applicant: ZEBRA TECHNOLOGIES CORPORATION, Lincolnshire, IL (US)

(72) Inventors: Aleksandar Rajak, Ottawa, CA (US); Alexander Miller, Ottawa (CA)

(21) Appl. No.: 16/774,604

(22) Filed: Jan. 28, 2020

(51) Int. Cl. A61B 5/00; G06T 7/00

(52) U.S. Cl. CPC A61B 5/441; G06T 2207/10028; G06T 2207/30096; G06T 7/0012

(57) ABSTRACT

A method of lesion monitoring includes: obtaining, at a computing device, image data and depth data depicting a lesion on a patient; detecting, at the computing device, a set of anchor points in at least one of the image data and the depth data; defining a frame of reference according to the anchor points; based on the frame of reference, detecting physical characteristics of the lesion from the image data and the depth data; and presenting the physical characteristics of the lesion.

### BACKGROUND

Tissue lesions such as external wounds and the like can change in appearance and structure over time, due to healing, progression of an underlying condition, or the like. Existing mechanisms for monitoring such changes are manual and therefore time-consuming and prone to error.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a diagram of a system for lesion monitoring.

FIG. 2 is a flowchart of a method for lesion monitoring.

FIG. 3 is a diagram illustrating an image captured at block 205 of the method of FIG. 2, and anchor points detected at block 210 of the method of FIG. 2.

FIG. 4A is a diagram illustrating a frame of reference defined at block 210 of the method of FIG. 2.

FIG. 4B is a diagram further illustrating the frame of reference of FIG. 4A.

FIG. 5A is a diagram illustrating a lesion boundary and dimensions detected at block 215 of the method of FIG. 2.

FIG. 5B is a diagram illustrating a lesion area detected at block 215 of the method of FIG. 2.

FIG. 6 is a diagram illustrating a lesion color profile and depth profile generated at block 220 of the method of FIG. 2.

FIG. 7 is a diagram illustrating an image captured at block 205 of the method of FIG. 2.

FIG. 8 is a diagram illustrating a comparison of the color profile of FIG. 6 with an additional color profile at block 230 of the method of FIG. 2.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Examples disclosed herein are directed to a method of lesion monitoring, comprising: obtaining, at a computing device, image data and depth data depicting a lesion on a patient; detecting, at the computing device, a set of anchor points in at least one of the image data and the depth data; defining a frame of reference according to the anchor points; based on the frame of reference, detecting physical characteristics of the lesion from the image data and the depth data; and presenting the physical characteristics of the lesion.

Additional examples disclosed herein are directed to a computing device for lesion monitoring, comprising: a memory; and a processor configured to: obtain image data and depth data depicting a lesion on a patient; detect, at the computing device, a set of anchor points in at least one of the image data and the depth data; define a frame of reference according to the anchor points; based on the frame of reference, detect physical characteristics of the lesion from the image data and the depth data; and present the physical characteristics of the lesion.

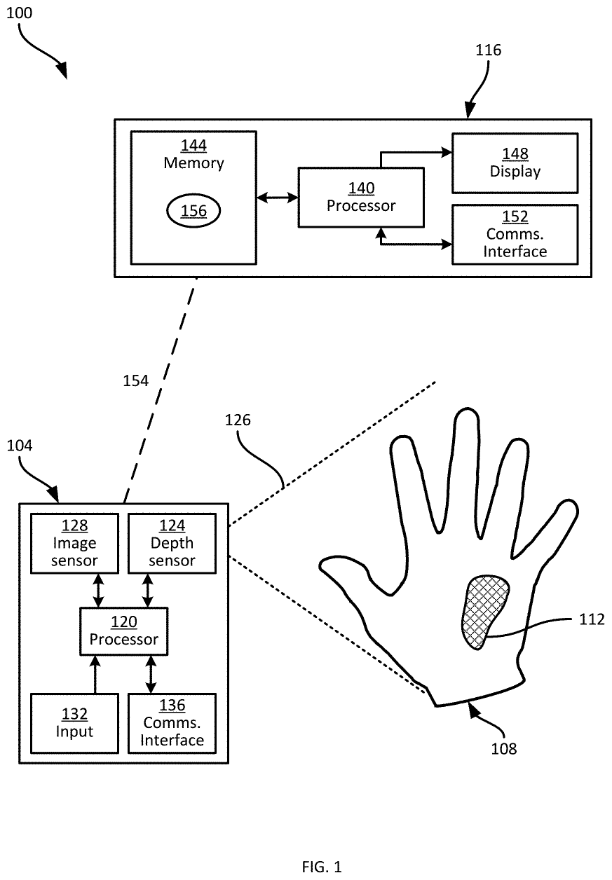

FIG. 1 depicts a system 100 for monitoring lesions, particularly externally visible lesions, on the skin of a patient. Patients (e.g. human patients, livestock and the like) may develop any of a wide variety of lesions, including wounds, ulcers, skin tumors, moles and the like. Treatment of a lesion can include recording physical characteristics of the lesion, at least once and in some cases more than once over the course of a period of time. The recorded characteristics can be used to assess the severity of a wound and/or healing of the wound over time, growth of a potentially malignant mole, and the like.

The system 100 enables at least partially automated capture of the above-mentioned physical characteristics, as well as at least partially automated comparison of physical characteristics of a given lesion with previously recorded physical characteristics of that same lesion. In particular, the system 100 includes a data capture device 104, such as a handheld computer or other mobile computing device. The data capture device 104 is used to capture image and/or depth data depicting patient tissue such as a hand 108 of a patient, bearing a lesion 112. As will be apparent throughout the discussion below, lesions on a wide variety of other patient tissues can also be recorded using the systems and methods described herein.

The data captured by the data capture device 104 (e.g. image and depth data) can be provided to a computing device 116 for processing and presentation and storage of output (e.g. the above-mentioned physical characteristics of the lesion 112). The computing device 116, for example, can determine the physical characteristics of the lesion 112, as well as store the physical characteristics and compare the physical characteristics to previously recorded characteristics of the lesion 112 to track healing progress or other changes to the lesion 112 over time.

The computing device 116 can be implemented as a laptop computer, a desktop computer, a server, or the like. In some examples, the computing device 116 and the data capture device 104 are implemented as a single computing device that is configured to both capture the image and depth data depicting the hand 108 and the lesion 112, and perform subsequent processing of the image and depth data.

Certain internal components of the data capture device 104 and the computing device 116 are also illustrated in FIG. 1. In particular, the device 104 includes a special-purpose controller, such as a processor 120, which may be interconnected with or include a non-transitory computer readable storage medium. The processor 120 and the above-mentioned memory can be implemented as at least one integrated circuit. In some examples, the processor 120 and at least a portion of the other components of the device 104 can be implemented on a single integrated circuit, e.g. as a system on a chip (SoC).

The device 104 also includes a depth sensor 124 such as a stereo camera configured to capture stereo images covering a field of view (FOV) 126 and generate, from the stereo images, a point cloud representing objects within the FOV 126. The depth sensor 124 can also be implemented as a time-of-flight (ToF) camera or other suitable depth-sensing mechanisms. The device 104 further includes an image sensor 128, such as a color camera, configured to capture a two-dimensional color image of the FOV 126. In other examples, the FOVs of the sensors 124 and 128 need not be identical as shown in FIG. 1.

The device 104 includes an input assembly 132 including any one or more of a touch screen, a keypad, a microphone or the like. The input assembly 132 can provide input to the processor 120 in response to activation, e.g. by an operator of the device 104. Upon receipt of the input, the processor 120 can control the depth sensor 124 and the image sensor 128 to capture depth data and image data, respectively, of the FOV 126. The captured data can be provided to the computing device 116 via a communications interface 136 of the device 104. The communications interface 136 can provide either or both of wireless (e.g. Wi-Fi or the like) and wired (e.g. Universal Serial Bus (USB)) connectivity to the computing device 116.

The computing device 116 includes a special-purpose controller, such as a processor 140, interconnected with a non-transitory computer readable storage medium, such as a memory 144. The memory 144 includes a combination of volatile memory (e.g. Random Access Memory or RAM) and non-volatile memory (e.g. read only memory or ROM, Electrically Erasable Programmable Read Only Memory or EEPROM, flash memory). The processor 140 and the memory 144 each comprise at least one integrated circuit.

The computing device 116 also includes an output assembly including, for example, a display 148. The output assembly can include a variety of other mechanisms, such as a speaker. The computing device 116 can also include an input assembly (not shown) for providing input data to the processor 140 representative of input from an operator of the computing device 116 (e.g. the same operator as that of the device 104, or a different operator). The computing device 116 also includes a communications interface 152 enabling the exchange of data, including the image and depth data mentioned above, with the data capture device 104. For example, the communications interfaces 152 and 136 can implement a link 154 between the computing device 116 and the data capture device 104.

The memory 144 stores computer readable instructions for execution by the processor 140. In particular, the memory 144 stores a lesion monitoring application 156 (also referred to simply as the application 156) which, when executed by the processor 140, configures the processor 140 to perform various functions discussed below in greater detail and related to detecting lesions in data captured with the sensors 124 and 128, extracting physical characteristics of the lesions from the captured data and subsequent processing of the physical characteristics. The application 156 may also be implemented as a suite of distinct applications in other examples.

Those skilled in the art will appreciate that the functionality implemented by the processor 140 via the execution of the application 156 may also be implemented by one or more specially designed hardware and firmware components, such as FPGAs, ASICs and the like in other embodiments. As noted above, in some examples the computing device 116 and the data capture device 104 are implemented as a single device. For example, the single computing device can include the sensors 124 and 128, the input 132, the display 148, the memory 144 (and the application 156 stored therein), as well as at least one processor and at least one communications interface.

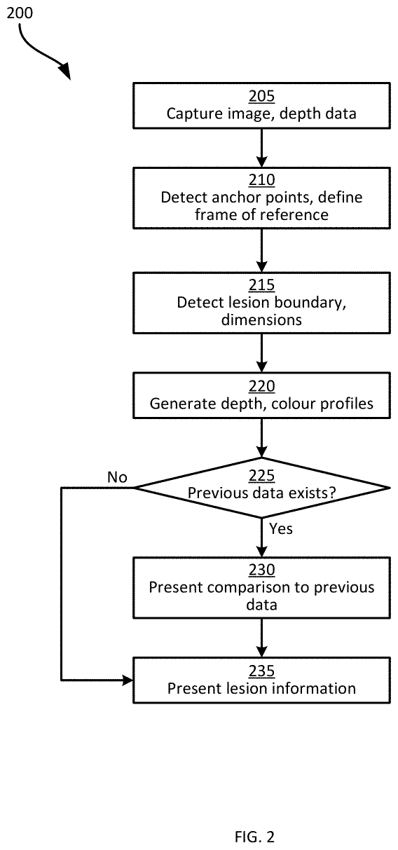

Turning now to FIG. 2, a method 200 for monitoring lesions is illustrated. The method 200 will be discussed below in conjunction with its performance in the system 100, but it will be apparent to those skilled in the art that the method 200 may also be performed by other systems equivalent to that shown in FIG. 1.

At block 205, the data capture device 104 is controlled to capture image and depth data depicting the lesion 112 on the hand 108. For example, the hand 108 can be placed within the FOV 126 of the sensors 124 and 128, and the input 132 can be activated to initiate a data capture operation. The sensor 124 can be controlled by the processor 120, in response to activation of the input 132, to capture a plurality of depth measurements representing the hand (or at least the portion thereof that is within the FOV 126) as a point cloud. The sensor 128 can be controlled by the processor 120, in response to the same activation of the input 132, to capture an image (e.g. a two-dimensional color image) of the portion of the hand 108 that appears within the FOV 126. The captured depth and image data can be registered to a common frame of reference, e.g. such that at least a subset of the pixels in the two-dimensional image are supplemented with a corresponding depth measurement.

The data capture device 104 can store the captured depth and image data, and can also transmit the captured data to the computing device 116 for further processing. In the discussion below, the computing device 116 is assumed to perform the remaining blocks of the method 200. In other examples, however, the data capture device 104 can perform at least a portion of the functions discussed below. In further examples, as mentioned earlier, the data capture device 104 and the computing device 116 are implemented as a single computing device and the blocks of the method 200 are therefore performed by that single computing device.

In some examples, prior to proceeding to block 210, the computing device 116 can be configured to segment the image to remove background pixels/voxels, such that the retained image and depth data represents only the portion of the hand 108 within the FOV 126.

At block 210 the computing device 116, having received the depth and image data from the data capture device 104, is configured via execution of the application 156 by the processor 140 to detect a set of anchor points in the data obtained at block 205. The anchor points, which may also be referred to as anatomical reference points, are positions in the captured data that are readily detectable in the current captured data as well as in future data captures also depicting the hand 108. The anchor points are also detectable at various orientations and scales (e.g. as a result of different distances between the sensors 124 and 128 and the hand 108).

The anchor points can be detected based on the image data, the depth data, or a combination thereof. For example, anchor points can be detected by detecting color gradients in the image data. In other examples, feature point (also referred to as keypoint) detection algorithms may also be applied to the image data at block 210, to detect edges, corners or the like. Examples of feature detection algorithms include the oriented FAST and rotated BRIEF (ORB) feature detector.

In some examples, the computing device 116 can receive a region indicator indicating a region of the patient represented by the captured data. For example, the region indicator can indicate that the captured data depicts a hand, a foot, an abdomen, or the like. The region indicator can be provided to the data capture device 104 via the input 132 for transmission to the computing device 116. In other examples, the region indicator can be provided directly to the computing device 116 via an input assembly thereof. In examples using a region indicator, the computing device 116 can select different criteria (e.g. a different feature detection algorithm) for use in detecting the anchor points.

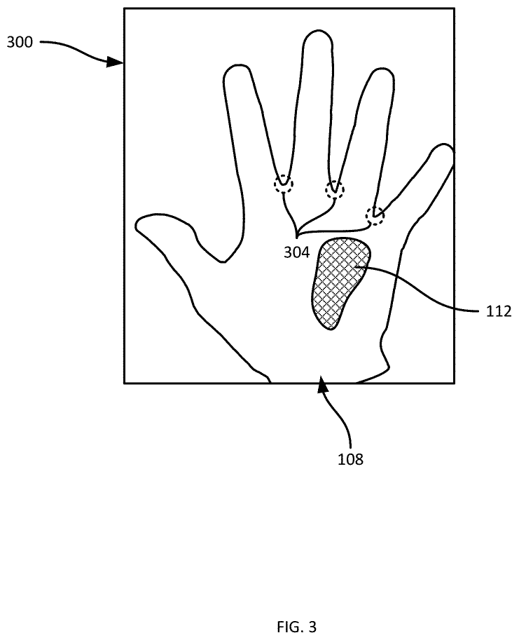

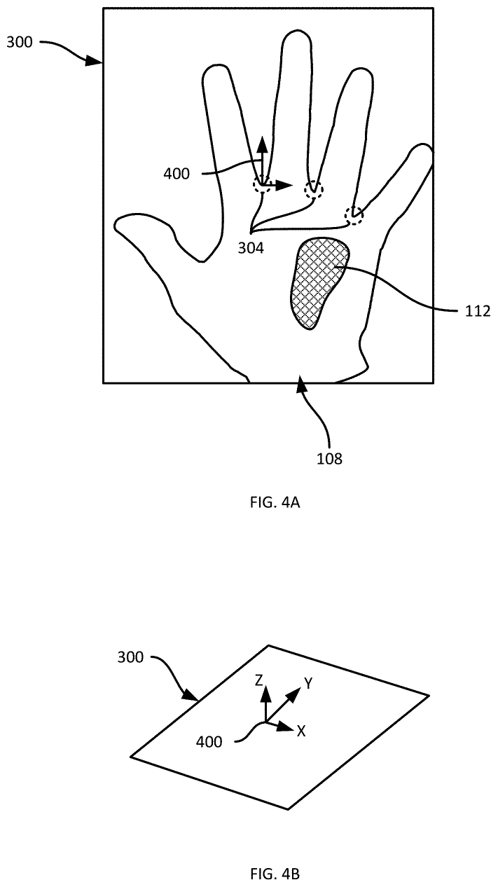

Turning to FIG. 3, an image 300 is shown as captured by the data capture device 104 and provided to the computing device 116. The image 300 depicts a portion of the hand 108, as well as the lesion 112. Also shown in FIG. 3, are three anchor points 304, corresponding to the webs between fingers of the hand 108 (which may also be referred to as interdigital folds). Having detected the anchor points 304, the computing device 116 defines a frame of reference based at least in part on the anchor points 304. For example, as shown in FIG. 4, a frame of reference 400 is defined with an origin at one of the anchor points 304. Two axes of the frame of reference 400, are shown in FIG. 4A, and a third axis is orthogonal to the axes shown in FIG. 4A. FIG. 4B illustrates a perspective view of a boundary of the image 300, along with the frame of reference 400. In particular, the frame of reference 400 includes X and Y axes (visible in FIG. 4A), and a Z axis orthogonal to the X and Y axes.

Although the frame of reference 400 is shown as having an origin at an anchor point 304, in other examples the origin of the frame of reference 400 can be placed at any other suitable location within the image 300. Following establishment of the frame of reference 400, the image and depth data can be registered to the frame of reference 400, such that each pixel and/or voxel is assigned coordinates based on its position relative to the origin of the frame of reference 400. The positions of the anchor points 304 in the frame of reference 400 are also stored at block 210. Storage of the positions of the anchor points 304 enables the computing device 116 to locate the origin of the frame of reference 400 in subsequent images of the same patient tissue, by locating the anchor points 304 and determining the location of the frame of reference 400 based on the stored positions.

Referring again to FIG. 2, at blocks 215 and 220 the computing device 116 is configured to detect various physical characteristics of the lesion 112. For example, at block 215 the computing device 116 can detect a boundary of the lesion 112. Detection of a lesion boundary can be performed by the computing device 116 based on any suitable image processing and/or depth processing mechanisms. For example, color gradients can be assessed in the image 300 according to a suitable one of, or a suitable combination of, edge detection and/or blob detection mechanisms to detect the boundary of the lesion 112 based on differences in color between the lesion 112 and the surrounding skin. In some examples, the depth data can also be processed to detect the lesion boundary, e.g. by searching for regions containing discontinuities such as changes in a surface profile of the hand 108 that exceed a threshold. Such discontinuities may indicate sudden peaks or depressions in the skin, indicative of the boundary of the lesion 112.

The position of the boundary of the lesion 112 can be stored in the memory 144, e.g. as a series of points in the frame of reference 400 that define the boundary. At block 210 the computing device 116 can also determine at least one dimension of the lesion 112. For example, the computing device 116 can detect, based on the boundary, a maximum dimension of the lesion in a plane parallel with the surrounding skin, and store the detected maximum dimension as a length. The computing device 116 can also detect a further maximum dimension (also in a plane parallel with the surrounding skin) that is orthogonal to the length, and store the further maximum dimension as a width. In addition, the computing device 116 can detect a maximum depth of the lesion 112, corresponding to a maximum dimension that is orthogonal to both the length and the width (and therefore is also orthogonal to the surrounding skin of the hand 108).

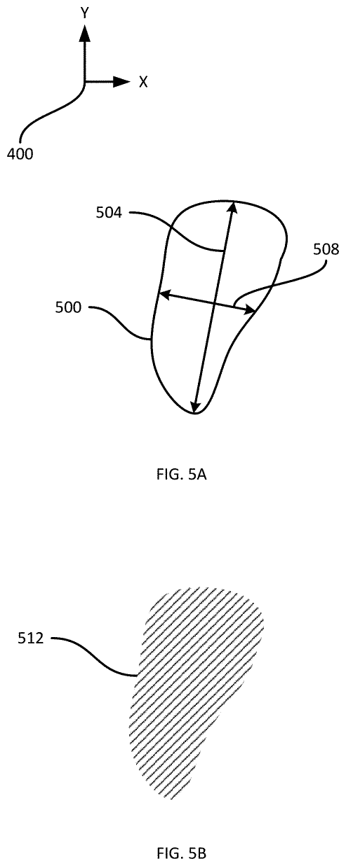

Turning to FIG. 5A, a boundary 500 of the lesion 112 is shown, along with a length 504 and a width 508 of the lesion 112. The length 504 and width 508 can be stored as scalar quantities (e.g. in millimeters or any other suitable unit of measurement). In some examples, the length 504 and width 508 can be stored along with coordinates in the frame of reference 400 indicating the locations to which the length 504 and width 508 correspond.

Various other dimensions can also be determined at block 215. For example as shown in FIG. 5B, an area 512 of the lesion 112 can be determined. The area 512 can correspond to the area within the boundary 500 shown in FIG. 5A. In some examples, a volume of the lesion 112 can also be determined, based on the boundary 500 and the depth of the lesion 112 within the boundary 500. A further example of dimensional information determined at block 215 can include coordinates of a center of the lesion 112 in the frame of reference 400.

The dimensions mentioned above can be stored in the memory 144, for example in association with at least one of a time and/or date, a patient identifier, a lesion identifier, and the like.

At block 220, the computing device 116 can generate additional physical characteristics of the lesion 112, in the form of either or both of a depth profile and a color profile of the lesion. The computing device 116 can, for example, store a set of predetermined color ranges. To generate the color profile, the computing device 116 can determine from the image data which pixels within the boundary 500 fall within each of the predetermined color ranges. Based on the sets of pixels that fall within each predetermined color range, the computing device 116 can generate sub-boundaries within the boundary 500, indicating regions of the lesion 112 with similar colors (which may, for example, be an indication of the depth and/or severity of the lesion 112).

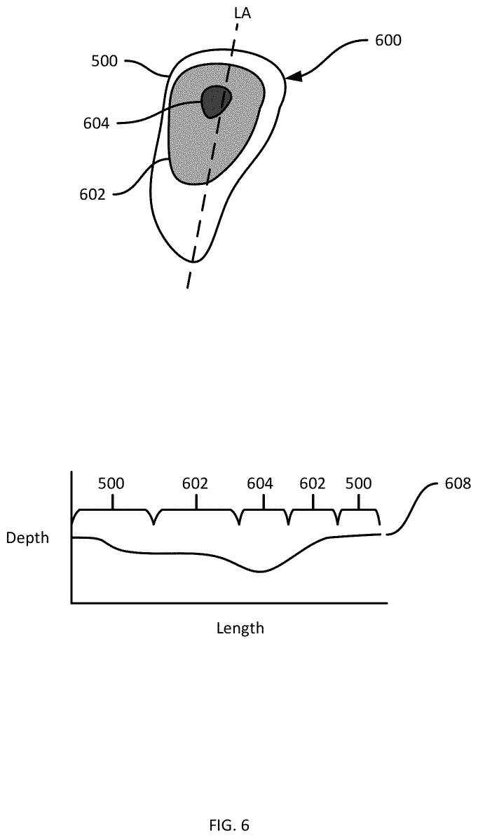

Turning to FIG. 6, a color profile 600 is shown in which the boundary 500 represents a first region of pixels in the image data that have colors within a given one of the above-mentioned color ranges. Additional boundaries 602 and 604 within the boundary 500 define additional regions of pixels in the image data that have colors within respective ones of the above-mentioned color ranges. The color profile 600 can be stored as an image, as coordinates in the frame of reference 400 defining the sub-boundaries 602 and 604, or a combination of the above.

A depth profile generated at block 220 can include a plot of the depth of the lesion 112 relative to the length 504 mentioned above. FIG. 6 illustrates a depth profile 608, which represents the depth of the lesion 112 relative to the length 504, as represented over the color profile 600 as a longitudinal axis “LA”. Brackets adjacent to the profile 608 indicate the color regions that correspond to respective portions of the profile 608. A width-wise depth profile may also be generated at block 220. As with the dimensions mentioned earlier, the color and/or depth profiles generated at block 220 can be stored in the memory 144 in association with the time and/or date, patient identifier, lesion identifier and the like.

Returning to FIG. 2, at block 225 the computing device 116 is configured to determine whether previous data corresponding to the lesion 112 is stored in the memory 144 or otherwise available to the computing device 116. In particular, the computing device 116 can query a database or other storage mechanism for other datasets having the same patient and/or lesion identifier as mentioned above, but an earlier time and/or date associated therewith. When the determination at block 225 is affirmative, the computing device 116 proceeds to block 230.

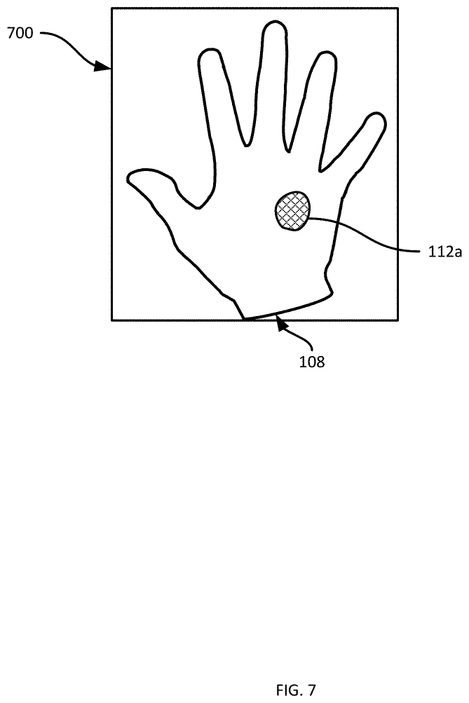

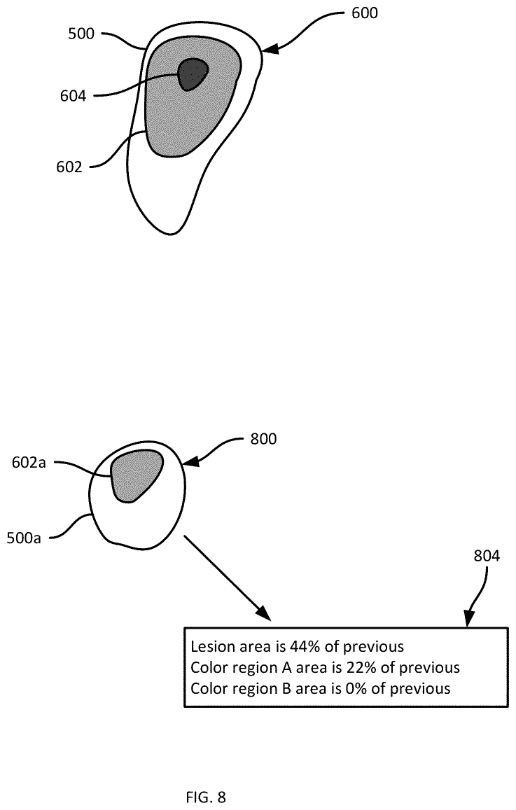

At block 230, the computing device 116 is configured to generate comparison data based on the physical characteristics detected at blocks 215 and 220, as well as on previous physical characteristics detected via earlier performances of blocks 215 and 220. For example, turning to FIG. 7, an example image 700 is shown of the hand 108, captured at a later time than the image 300 shown in FIG. 3. As seen in the image 300, the hand 108 bears a lesion 112a, which occupies a smaller area on the hand 108 than the lesion 112 (e.g. because the lesion 112 has partially healed). FIG. 8 illustrates the color profile 600 mentioned earlier, as well as a color profile 800 generated from the image 700 in a subsequent performance of the method 200. As will be apparent, following generation of the color profile 800 (and determination of other physical characteristics of the lesion 112a at blocks 215 and 220), the determination at block 225 is affirmative, because the color profile 600 was generated and stored previously.

At block 230, therefore, the computing device 116 can generate comparison data based on at least some of the physical characteristics and the corresponding previously determined characteristics. Thus, the color profile 800 can be compared with the color profile 600. The comparison data 804 can include, for example, a ratio of an area within the boundary 500a of the lesion 112a with the area of the boundary 500 (e.g. 44% in the illustrated example). The comparison data 804 can also include a ratio of the area of the internal boundary 602a to the previous internal boundary 602 (e.g. 22% in the illustrated example). A similar comparison for the previous internal boundary 604 yields a ratio of zero, as the color profile 800 indicates that the image 700 does not contain any pixels within the predefined color range corresponding to the internal boundary 604.

Various other forms of comparison data can also be generated by the computing device 116, including changes in dimensions and position of the lesion 112, and the like. The comparison data 804 can be stored in the memory 144, e.g. for rendering on the display 148, transmission to another computing device (including the device 104), or a combination thereof.

Returning again to FIG. 2, when the comparison at block 230 has been generated, or simultaneously with generation of the comparison, at block 235 the computing device 116 is configured to present the lesion information from blocks 215 and 220. As shown in FIG. 2, the computing device 116 also proceeds directly to block 235 from block 225 when no previous information for the lesion 112 is available. The performance of block 235 can include rendering the data from blocks 215 and 220 simultaneously with the comparison data (e.g. the comparison data 804 mentioned above) from block 230. The performance of block 235 can also include the transmission of the lesion information to another computing device for rendering at that device.

The performance of the method 200 may be repeated, for other lesions and/or for the same lesion as discussed above. When more than one previous capture exists for a given lesion, the comparison data generated at block 230 can be based not only on the immediately preceding lesion information, but on as many as all sets of previous lesion information for the relevant lesion. Thus, the comparison data can illustrate a progression of a lesion over time, including as many steps in the progression as there have been performances of the method 200 for that lesion.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

It will be appreciated that some embodiments may be comprised of one or more specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A method of lesion monitoring, comprising:
obtaining, at a computing device, image data and depth data depicting a lesion on a patient;
detecting, at the computing device, a set of anchor points in at least one of the image data and the depth data;
defining a frame of reference according to the anchor points;
based on the frame of reference, detecting physical characteristics of the lesion from the image data and the depth data; and
presenting the physical characteristics of the lesion.

2. The method of claim 1, wherein obtaining the image data and depth data includes capturing the image and depth data via a data capture device including image and depth sensors.

3. The method of claim 2, wherein obtaining the image data and depth data further includes receiving the data at the computing device from the data capture device.

4. The method of claim 1, wherein the anchor points correspond to anatomical features.

5. The method of claim 4, further comprising:
receiving input indicating a region of the patient depicted in the image data and the depth data; and
detecting the anchor points according to the region.

6. The method of claim 1, wherein the physical characteristics include a lesion boundary, and at least one lesion dimension based on the boundary.

7. The method of claim 1, wherein presenting the physical characteristics includes rendering the physical characteristics on a display.

8. The method of claim 1, further comprising:
receiving a patient identifier associated with the image data and the depth data;
determining whether previous physical characteristics of the lesion are stored; and
when the determination is affirmative, generating comparison data based on the physical characteristics and the previous physical characteristics of the lesion.

9. The method of claim 8, further comprising presenting the comparison data.

10. The method of claim 9, further comprising presenting the comparison data simultaneously with the physical characteristics of the lesion.

11. A computing device for lesion monitoring, comprising:
a memory; and
a processor configured to:
obtain image data and depth data depicting a lesion on a patient;
detect, at the computing device, a set of anchor points in at least one of the image data and the depth data;
define a frame of reference according to the anchor points;
based on the frame of reference, detect physical characteristics of the lesion from the image data and the depth data; and
present the physical characteristics of the lesion.

12. The computing device of claim 11, further comprising:
an image sensor and a depth sensor;
wherein the processor is configured, in order to obtain the image data and depth data, to control the image sensor and the depth sensor to capture the image data and the depth data.

13. The computing device of claim 11, wherein the processor is configured, in order to obtain the image data and the depth data, to receive the data from a data capture device.

14. The computing device of claim 11, wherein the anchor points correspond to anatomical features.

15. The computing device of claim 14, wherein the processor is further configured to:
receive input indicating a region of the patient depicted in the image data and the depth data; and
detect the anchor points according to the region.

16. The computing device of claim 11, wherein the physical characteristics include a lesion boundary, and at least one lesion dimension based on the boundary.

17. The computing device of claim 11, further comprising:
a display;
wherein the processor is further configured, in order to present the physical characteristics, to render the physical characteristics on the display.

18. The computing device of claim 11, wherein the processor is further configured to:
receive a patient identifier associated with the image data and the depth data;
determine whether previous physical characteristics of the lesion are stored; and
when the determination is affirmative, generate comparison data based on the physical characteristics and the previous physical characteristics of the lesion.

19. The computing device of claim 18, wherein the processor is further configured to present the comparison data.

20. The computing device of claim 19, wherein the processor is further configured to present the comparison data simultaneously with the physical characteristics of the lesion.
